(19) United States
(12) Patent Application Publication
(10) Pub. No.: US 2010/0119460 A1
(43) Pub. Date: May 13, 2010

(54) Azeotrope-Like Compositions Of 2,3,3,3-Tetrafluoropropene And 3,3,3-Trifluoropropene

(75) Inventors: HANG T. PHAM, Amherst, NY (US); RAJIV R. SINGH, Getzville, NY (US); HSUEH S. TUNG, Getzville, NY (US); DANIEL C. MERKEL, West Seneca, NY (US); HALUK KOPKALLI, Staten Island, NY (US); RYAN HULSE, Getzville, NY (US)

Correspondence Address: HONEYWELL INTERNATIONAL INC.;PATENT SERVICES, 101 COLUMBIA ROAD, P O BOX 2245, MORRISTOWN, NJ, 07962-2245, US

(73) Assignee: HONEYWELL INTERNATIONAL INC., Morristown, NJ (US)

(21) Appl. No.: 12/614,944

(22) Filed: Nov. 9, 2009

Related U.S. Application Data
(60) Provisional application No. 61/113,477, filed on Nov. 11, 2008.

(51) Int. Cl. A61K 9/12; C09K 5/04; C09K 3/14; C11D 7/50; C09K 21/08; C09J 11/06; C08J 9/14; C09K 3/00

(52) U.S. Cl. 424/47; 252/67; 252/68; 51/293; 424/45; 510/461; 252/605; 106/287.28; 521/98; 252/182.15; 252/182.12

(57) ABSTRACT

Provided are azeotropic or azeotrope-like mixtures of 2,3,3,3-tetrafluoropropene (1234yf) and 3,3,3-trifluoropropene (1243zf), as well as methods for producing and using the same.

### CROSS REFERENCE TO RELATED APPLICATION

This application claims the priority benefit of U.S. Provisional Application No. 61/113,477, filed Nov. 11, 2008, which is incorporated herein by reference.

### BACKGROUND OF THE INVENTION

1. Field of Invention

The present invention relates to azeotrope-like compositions. More particularly, the present invention relates binary azeotrope-like compositions of hydrofluoroolefins.

2. Description of Prior Art

Hydrofluorocarbons (HFCs), i.e., hydrofluoroalkanes, have many of the same physical properties as chlorofluorocarbons (CFCs) and hydrochlorofluorocarbons (HCFCs), particularly properties relevant to applications such as blowing agents, refrigerants, cleaning agents, aerosol propellants, heat transfer media, dielectrics, fire extinguishing compositions, power cycle working fluids, and the like. Unlike CFCs and HCFCs, however, HFCs have relatively little impact on atmospheric ozone. Accordingly, HFCs are used as environmentally friendly substitutes for CFCs and HCFCs in many commercial and/or industrial applications. Yet there is some evidence that HFCs might contribute to global warming. Consequently, it is desirable to find replacement molecules for HFCs that have short atmospheric lifetime and, thus, do not persist in the atmosphere thereby minimizing their overall global warming potential.

One such compound, 2,3,3,3-tetrafluoropropene (HFO-1234yf), has a relatively low global warming potential and could be a replacement for certain fluorocarbons, such as 1,1,1,2-tetrafluoroethane, in refrigeration systems, as a blowing agent, and in other commercial applications as well.

Many azeotropes possess properties that make them useful as refrigerants, blowing agents, propellants, solvents, and the like. For example, azeotropes have a constant boiling point that avoids boiling temperature drift during processing and use. Azeotropes used as propellants maintain a consistent composition even as the propellant is depleted from its source. Moreover, azeotropic propellants often have advantageous properties compared to single compound propellants, such as lower flammability, modified operating temperature, lower cost, and the like. When used as a solvent, azeotropes demonstrate constant physical properties because the composition of the solvent does not change during boiling or reflux. Azeotropes that are used as solvents also can be recovered conveniently by distillation.

However, the identification of new, environmentally-safe, non-fractionating mixtures that are commercially useful is complicated due to the fact that azeotrope formation is not readily predictable. Therefore, industry is continually seeking new azeotrope and azeotrope-like mixtures, particularly for combinations of compounds having low Global Warming Potential (GWP). This invention satisfies this need among others.

### SUMMARY OF INVENTION

Certain mixtures of 2,3,3,3-tetrafluoropropene (HFO-1234yf) and 3,3,3-trifluoropropene (HFO-1243zf) have been found to possess azeotrope and/or azeotrope-like properties. Such azeotrope and azeotrope-like compositions are useful as refrigerants, blowing agents and solvents compositions.

Accordingly, provided is an azeotrope-like mixture comprising effective amounts of 2,3,3,3-tetrafluoropropene and 3,3,3-trifluoropropene, preferably from about 60 to less than 100 weight percent 2,3,3,3-tetrafluoropropene and from greater than 0 to about 40 weight percent 3,3,3-trifluoropropene, more preferably from about 85 to less than 100 weight percent 2,3,3,3-tetrafluoropropene and from greater than 0 to about 15 weight percent 3,3,3-trifluoropropene, even more preferably from about 95 to less than 100 weight percent 2,3,3,3-tetrafluoropropene and from greater than 0 to about 5 weight percent 3,3,3-trifluoropropene, and most preferably from about 95.8 to about 99.9 weight percent 2,3,3,3-tetrafluoropropene and from greater than 0.1 to about 4.2 weight percent 3,3,3-trifluoropropene. In another aspect of the invention, provided is a method for preparing an azeotrope-like composition comprising blending 3,3,3-trifluoropropene and 2,3,3,3-tetrafluoropropene to produce a composition comprising from about 60 to less than 100 weight percent 2,3,3,3-tetrafluoropropene and from greater than 0 to about 40 weight percent 3,3,3-trifluoropropene, and maintaining said composition at a temperature of about −30° C. to about 66° C. and at a pressure of about 14 psia to about 230 psia.

In another aspect of the invention, provided is a process for the synthesis of 1234yf via the steps of hydrogenating 1225ye to produce 245eb and 254eb (minor over hydrogenated co-product) followed by dehydrofluorination of said 245eb and 254eb stream using a caustic solution or a bulk or supported vapor phase dehydrofluorination catalyst. Examples of dehydrofluorination catalysts are fluorinated Cr2O3, A1F3, activated carbon, CsC12/MgO, CsC12/MgF2. Said dehydrofluorination reaction produces 1234yf and 1243zf as a minor co-product.

In yet another aspect of the invention, provided is a method for purifying a hydrofluoroolefin comprising: (a) providing a composition comprising 2,3,3,3-tetrafluoropropene and 3,3,3-trifluoropropene; and (b) distilling said composition to produce a first stream enriched in either said 2,3,3,3-tetrafluoropropene or said 3,3,3-trifluoropropene and a second stream comprising an azeotrope-like mixture of 2,3,3,3-tetrafluoropropene and 3,3,3-trifluoropropene; and optionally, (c) breaking said azeotrope-like mixture by subjecting said azeotrope-like mixture to at least one separation method selected from the group consisting of swing distillation, extractive distillation, azeotrope distillation, evaporation, and phase separation; and (d) separating said broken azeotrope-like mixture into a first component rich in 2,3,3,3-tetrafluoropropene and a second component rich in 3,3,3-trifluoropropene.

Another aspect of the invention provides a blowing agent comprising an azeotrope-like mixture of 2,3,3,3-tetrafluoropropene and 3,3,3-trifluoropropene, and, optionally, co-blowing agents, fillers, vapor pressure modifiers, flame suppressants, and stabilizers.

Another aspect of the invention provides a sprayable composition comprising an azeotrope-like mixture of 2,3,3,3-tetrafluoropropene and 3,3,3-trifluoropropene, an active ingredient, and, optionally, inert ingredients and/or solvents and aerosol propellants.

Yet another aspect of the invention provides a closed cell foam comprising a polyurethane-, polyisocyanurate-, or phenolic-based cell wall and a cell gas disposed within at least a portion of the cell wall structure, wherein the cell gas comprises an azeotrope-like mixture of 2,3,3,3-tetrafluoropropene and 3,3,3-trifluoropropene.

Another aspect of the invention provides a polyol premix comprising an azeotrope-like of 2,3,3,3-tetrafluoropropene and 3,3,3-trifluoropropene.

Another aspect of the invention provides a foamable composition comprising an azeotrope-like of 2,3,3,3-tetrafluoropropene and 3,3,3-trifluoropropene.

Another aspect of the invention provides a method for producing thermoset foam comprising (a) adding a blowing agent comprising an azeotrope-like of 2,3,3,3-tetrafluoropropene and 3,3,3-trifluoropropene to a foamable mixture comprising a thermosetting resin; (b) reacting said foamable mixture to produce a thermoset foam; and (c) volatilizing said azeotrope-like composition during said reacting.

Another aspect of the invention provides a method for producing thermoplastic foam comprising (a) adding a blowing agent comprising an azeotrope-like of 2,3,3,3-tetrafluoropropene and 3,3,3-trifluoropropene to a foamable mixture comprising a thermoplastic resin; (b) reacting said foamable mixture to produce a thermoplastic foam; and (c) volatilizing said azeotrope-like composition during said reacting.

Another aspect of the invention provides a thermoplastic foam having a cell wall comprising a thermoplastic polymer and a cell gas comprising an azeotrope-like mixture of 2,3,3,3-tetrafluoropropene and 3,3,3-trifluoropropene.

Another aspect of the invention provides a thermoset foam having a cell wall comprising a thermosetting polymer and a cell gas comprising an azeotrope-like mixture of 2,3,3,3-tetrafluoropropene and 3,3,3-trifluoropropene.

In yet another aspect of the invention, provided is a solvent comprising an azeotrope-like mixture of 2,3,3,3-tetrafluoropropene and 3,3,3-trifluoropropene.

In yet another aspect of the invention, provided is method for replacing an existing refrigerant contained in a refrigerant system comprising removing at least a portion of said existing refrigerant from said system and replacing at least a portion of said existing refrigerant by introducing into said system a new refrigerant composition comprising an azeotrope-like mixture of 2,3,3,3-tetrafluoropropene and 3,3,3-trifluoropropene.

### DETAILED DESCRIPTION OF PREFERRED EMBODIMENTS

According to certain embodiments, the present invention provides azeotrope-like compositions comprising, and preferably consisting essentially of, 2,3,3,3-tetrafluoropropene (HFO-1234yf) and 3,3,3-trifluoropropene (HFO-1243zf), as well as methods of making and using the same. Thus, the present invention overcomes the aforementioned shortcomings by providing azeotrope-like compositions that are, in preferred embodiments, substantially free of CFCs, HCFCs, and HFCs, and have very low global warming potentials, do not contribute to ozone depletion, and exhibit relatively constant boiling point and vapor pressure characteristics.

### Azeotrope-Like Compositions:

In certain embodiments, compositions of the invention comprise azeotrope-like mixtures of HFO-1234yf and HFO-1243zf. In certain embodiments, azeotrope-like compositions are provided that comprise effective amounts of 2,3,3,3-tetrafluoropropene and 3,3,3-trifluoropropene, preferably consists essentially of 2,3,3,3-tetrafluoropropene and 3,3,3-trifluoropropene, more preferably consist essentially of about 60 to less than 100 weight percent 2,3,3,3-tetrafluoropropene and from greater than 0 to about 40 weight percent 3,3,3-trifluoropropene, more preferably from about 85 to less than 100 weight percent 2,3,3,3-tetrafluoropropene and from greater than 0 to about 15 weight percent 3,3,3-trifluoropropene, even more preferably from about 95 to less than 100 weight percent 2,3,3,3-tetrafluoropropene and from greater than 0 to about 5 weight percent 3,3,3-trifluoropropene, and most preferably from about 95.8 to about 99.9 weight percent 2,3,3,3-tetrafluoropropene and from greater than 0.1 to about 4.2 weight percent 3,3,3-trifluoropropene.

As used herein, the term “azeotrope-like” relates to compositions that are strictly azeotropic or that generally behave like azeotropic mixtures. An azeotropic mixture is a system of two or more components in which the liquid composition and vapor composition are equal at the stated pressure and temperature. In practice, this means that the components of an azeotropic mixture are constant-boiling or essentially constant-boiling and generally cannot be thermodynamically separated during a phase change. The vapor composition formed by boiling or evaporation of an azeotropic mixture is identical, or substantially identical, to the original liquid composition. Thus, the concentration of components in the liquid and vapor phases of azeotrope-like compositions change only minimally, if at all, as the composition boils or otherwise evaporates. In contrast, boiling or evaporating non-azeotropic mixtures changes the component concentrations in the liquid phase to a significant degree.

As used herein, the term “consisting essentially of”, with respect to the components of an azeotrope-like composition, means the composition contains the indicated components in an azeotrope-like ratio, and may contain additional components provided that the additional components do not form new azeotrope-like systems. For example, azeotrope-like mixtures consisting essentially of two compounds are those that form binary azeotropes, which optionally may include one or more additional components, provided that the additional components do not render the mixture non-azeotropic and do not form an azeotrope with either or both of the compounds.

The term “effective amounts” as used herein refers to the amount of each component which, upon combination with the other component, results in the formation of an azeotrope-like composition of the present invention.

In certain preferred embodiments, these azeotrope-like compositions have a boiling point of from about −30° C. to about 66° C. at a pressure ranging from about 14 psia to about 230 psia. In certain preferred embodiments, the azeotrope-like composition is maintained at a pressure of about 14.3±0.5 psia, while in other preferred embodiments the azeotrope-like composition is maintained at a pressure of from about 162.5±0.5 psia to about 166.6±0.5 psia, and in still other preferred embodiments the azeotrope-like composition is maintained at a pressure of from about 231.1±0.5 psia to about 231.9±0.5 psia.

The azeotrope-like compositions of the present invention may further include a variety of optional additives including, but not limited to, lubricants, stabilizers, metal passivators, corrosion inhibitors, flammability suppressants, and the like. Examples of suitable stabilizers include diene-based compounds, and/or phenol compounds, and/or epoxides selected from the group consisting of aromatic epoxides, alkyl epoxides, alkenyl epoxides, and combinations of two or more thereof. Preferably, these optional additives do not affect the basic azeotrope-like characteristic of the composition.

The azeotrope-like compositions of the present invention can be produced by combining effective amounts of 1234yf and 1243zf. Any of a wide variety of methods known in the art for combining two or more components to form a composition can be adapted for use in the present methods to produce an azeotrope-like composition. For example, 1234yf and 1243zf can be mixed, blended, or otherwise contacted by hand and/or by machine, as part of a batch or continuous reaction and/or process, or via combinations of two or more such steps. In light of the disclosure herein, those of skill in the art will be readily able to prepare azeotrope-like compositions according to the present invention without undue experimentation.

The azeotrope-like compositions of the present invention can be produced in the process for the synthesis of 1234yf via the steps of hydrogenating 1225ye to produce 245eb and 254eb (minor over hydrogenated co-product) followed by dehydrofluorination of said 245eb and 254eb stream using a caustic solution (with the addition of a phase transfer catalyst such as Aliquot 336) or a bulk or supported vapor phase dehydrofluorination catalyst. Examples of dehydrofluorination catalysts are fluorinated Cr2O3, A1F3, activated carbon, CsC12/MgO, CsC12/MgF2. Said dehydrofluorination reaction produces a product stream consisting of 1234yf and 1243zf as a minor co-product.

In one embodiment of the present invention, the azeotrope-like compositions of the present invention, co-product in the production of 1234yf, can be isolated by conventional distillation. As part of the process to produce 1234yf, a stream containing both components, enriched in 1234yf, is fed into a conventional distillation column where an azeotrope-like composition of the present invention is taken as the distillate stream from the top of the column and essentially pure 1234yf is taken from the bottom of the column. Essentially pure 1234yf means containing little of or no 1243zf. If a stream is enriched in 1243zf it can be distilled to obtain azeotropic composition and relatively pure 1243zf. The azeotropic composition could then be further purified to relatively pure 1234yf by pressure swing distillation, extractive methods or by other means known in the art.

### Uses of the Compositions

The present compositions have utility in a wide range of applications. For example, one embodiment of the present invention relates to blowing agent, aerosol and cleaning, and refrigerant compositions comprising the present azeotrope-like compositions.

### Blowing Agents:

In another embodiment of the invention, provided are blowing agents comprising at least one azeotrope-like mixture described herein. In respect to the preparation of polymer foams comprising the blowing agent described herein, and of the polymers and methods used to prepare these foams can be employed. Specifically, polymer foams are generally of two general classes, thermoplastic foams and thermoset foams.

Thermoplastic foams are produced generally via any method known in the art, including those described in Throne, Thermoplastic Foams, 1996, Sherwood Publishers, Hinkley, Ohio. or Klempner and Sendijarevic, Polymeric Foams and Foam Technology, 2^{nd} Edition 2004, Hander Gardner Publications. Inc, Cincinnati, Ohio. For example, extruded thermoplastic foams can be prepared by an extrusion process whereby a solution of blowing agent in molten polymer, formed in an extruded under pressure, is forced through an orifice onto a moving belt at ambient temperature or pressure or optionally at reduced pressure to aid in foam expansion. The blowing agent vaporizes and causes the polymer to expand. The polymer simultaneously expands and cools under conditions that give it enough strength to maintain dimensional stability at the time corresponding to maximum expansion. Polymers used for the production of extruded thermoplastic foams include, but are not limited to, polystyrene, polyethylene (HDPE, LDPE, and LLDPE), polypropylene, polyethylene terephthalate, ethylene vinyl acetate, and mixtures thereof. A number of additives are optionally added to the molten polymer solution to optimize foam processing and properties including, but not limited to, nucleating agents (e.g., talc), flame retardants, colorants, processing aids (e.g., waxes), cross linking agents, permeability modifiers, and the like. Additional processing steps such as irradiation to increase cross linking, lamination of a surface film to improve foam skin quality, trimming and planning to achieve foam dimension requirements, and other processes may also be included in the manufacturing process.

In general, the blowing agent may include the azeotrope-like compositions of the present invention in widely ranging amounts. It is generally preferred, however, that the blowing agents comprise at least about 15% by weight of the blowing agent. In certain preferred embodiments, the blowing agent comprises at least about 50% by weight of the present compositions, and in certain embodiments the blowing agent consists essentially of the present azeotrope-like composition. In certain preferred embodiments, the blowing agent includes, in addition to the present azeotrope-like mixtures, one or more co-blowing agents, fillers, vapor pressure modifiers, flame suppressants, stabilizers, and like adjuvants.

In certain preferred embodiments, the blowing agent is characterized as a physical (i.e., volatile) blowing agent comprising the azeotrope-like mixture of the present invention. In general, the amount of blowing agent present in the blended mixture is dictated by the desired foam densities of the final foams products and by the pressure and solubility limits of the process. For example, the proportions of blowing agent in parts by weight can fall within the range of about 1 to about 45 parts, more preferably from about 4 to about 30 parts, of blowing agent per 100 parts by weight of polymer. The blowing agent may comprise additional components mixed with the azeotrope-like composition, including chlorofluorocarbons such as trichlorofluoromethane (CFC-11), dichlorodifluoromethane (CFC-12), hydrochlorofluorocarbons such as 1,1-dichloro-1-fluoroethane (HCFC-141b), 1-chloro-1,1-difluoroethane (HCFC-142b), chlorodifluoromethane (HCFC-22), hydrofluorocarbons such as 1,1,1,2-tetrafluoroethane (HFC-134a), 1,1-difluoroethane (HFC-152a), 1,1,1,3,3-pentafluoropropane (HFC-245fa), and 1,1,1,3,3-pentafluorobutane (HFC-365mfc), hydrocarbons such as propane, butane, isobutane, cyclopentane, carbon dioxide, chlorinated hydrocarbons alcohols, ethers, ketones and mixtures thereof.

In certain embodiments, the blowing agent is characterized as a chemical blowing agents. Chemical blowing agents are materials that, when exposed to temperature and pressure conditions in the extruder, decompose to liberate a gas, generally carbon dioxide, carbon monoxide, nitrogen, hydrogen, ammonia, nitrous oxide, of mixtures thereof. The amount of chemical blowing agent present is dependent on the desired final foam density. The proportions in parts by weight of the total chemical blowing agent blend can fall within the range of from less than 1 to about 15 parts, preferably from about 1 to about 10 parts, of blowing agent per 100 parts by weight of polymer.

In certain preferred embodiments, dispersing agents, cell stabilizers, surfactants and other additives may also be incorporated into the blowing agent compositions of the present invention. Surfactants are optionally, but preferably, added to serve as cell stabilizers. Some representative materials are sold under the names of DC-193, B-8404, and L-5340 which are, generally, polysiloxane polyoxyalkylene block co-polymers such as those disclosed in U.S. Pat. Nos. 2,834,748, 2,917,480, and 2,846,458, each of which is incorporated herein by reference. Other optional additives for the blowing agent mixture include flame retardants or suppressants such as tri(2-chloroethyl)phosphate, tri(2-chloropropyl)phosphate, tri(2,3-dibromopropyl)-phosphate, tri(1,3-dichloropropyl) phosphate, diammonium phosphate, various halogenated aromatic compounds, antimony oxide, aluminum trihydrate, polyvinyl chloride, and the like.

With respect to thermoset foams, in general any thermoset polymer can be used, including but not limited to polyurethane, polyisocyanurate, phenolic, epoxy, and combinations thereof. In general these foams are produces by bringing together chemically reactive components in the presence of one or more blowing agents, including the azeotrope-like composition of this invention and optionally other additives, including but no limited to cell stabilizers, solubility enhancers, catalysts, flame retardants, auxiliary blowing agents, inert fillers, dyes, and the like.

With respect to the preparation of polyurethane or polyisocyanurate foams using the azeotrope like compositions described in the invention, any of the methods well known in the art can be employed, see Saunders and Frisch, Volumes I and II Polyurethanes Chemistry and technology, 1962, John Wiley and Sons, New York, N.Y. In general, polyurethane or polyisocyanurate foams are prepared by combining an isocyanate, a polyol or mixture of polyols, a blowing agent or mixture of blowing agents, and other materials such as catalysts, surfactants, and optionally, flame retardants, colorants, or other additives.

It is convenient in many applications to provide the components for polyurethane or polyisocyanurate foams in preblended formulations. Most typically, the foam formulation is preblended into two components. The isocyanate and optionally certain surfactants and blowing agents comprise the first component, commonly referred to as the “A” component. The polyol or polyol mixture, surfactant, catalysts, blowing agents, flame retardant, and other isocyanate reactive components comprise the second component, commonly referred to as the “B” component. Accordingly, polyurethane or polyisocyanurate foams are readily prepared by bringing together the A and B side components either by hand mix for small preparations and, preferably, machine mix techniques to form blocks, slabs, laminates, pour-in-place panels and other items, spray applied foams, froths, and the like. Optionally, other ingredients such as fire retardants, colorants, auxiliary blowing agents, water, and even other polyols can be added as a third stream to the mix head or reaction site. Most conveniently, however, they are all incorporated into one B Component as described above.

Any organic polyisocyanate can be employed in polyurethane or polyisocyanurate foam synthesis inclusive of aliphatic and aromatic polyisocyanates. Preferred as a class are the aromatic polyisocyanates. Typical aliphatic polyisocyanates are alkylene diisocyanates such as tri, tetra, and hexamethylene diisocyanate, isophorene diisocyanate, 4, 4′-methylenebis(cyclohexyl isocyanate), and the like; typical aromatic polyisocyanates include m-, and p-phenylene disocyanate, polymethylene polyphenyl isocyanate, 2,4- and 2,6-toluenediisocyanate, dianisidine diisocyanate, bitoylene isocyanate, naphthylene 1,4-diisocyanate, bis(4-isocyanatophenyl)methene, bis(2-methyl-4-isocyanatophenyl)methane, and the like.

Preferred polyisocyanates are the polymethylene polyphenyl isocyanates, particularly the mixtures containing from about 30 to about 85 percent by weight of methylenebis(phenyl isocyanate) with the remainder of the mixture comprising the polymethylene polyphenyl polyisocyanates of functionality higher than 2.

Typical polyols used in the manufacture of polyurethane foams include, but are not limited to, aromatic amino-based polyether polyols such as those based on mixtures of 2,4- and 2,6- toluenediamine condensed with ethylene oxide and/or propylene oxide. These polyols find utility in pour-in-place molded foams. Another example is aromatic alkylamino-based polyether polyols such as those based on ethoxylated and/or propoxylated aminoethylated nonylphenol derivatives. These polyols generally find utility in spray applied polyurethane foams. Another example is sucrose-based polyols such as those based on sucrose derivatives and/or mixtures of sucrose and glycerin derivatives condensed with ethylene oxide and/or propylene oxide.

Examples of polyols used in polyurethane modified polyisocyanurate foams include, but are not limited to, aromatic polyester polyols such as those based on complex mixtures of phthalate-type or terephthalate-type esters formed from polyols such as ethylene glycol, diethylene glycol, or propylene glycol. These polyols are used in rigid laminated boardstock, can be blended with other types of polyols such as sucrose based polyols, and used in other polyurethane foam applications such as described above.

Catalysts used in the manufacture of polyurethane foams are typically tertiary amines including, but not limited to, N-alkylmorpholines, N-alkylalkanolamines, N,N-dialkylcyclohexylamines, and alkylamines where the alkyl groups are methyl, ethyl, propyl, butyl, and the like and isomeric forms thereof; and hetrocyclic amines. Typical, but not limiting examples are triethylenediamine, tetramethylethylenediamine, bis(2-dimethylaminoethyl)ether, triethylamine, tripropylamine, tributylamine, triamylamine, pyridine, quinoline, dimethylpiperazine, piperazine, N,N-dimethylcyclohexylamine, N-ethylmorpholine, 2-methylpiperazine, N,N-dimethylethanolamine, tetramethylpropanediamine, methyltriethylenediamine, and the like, and mixtures thereof.

Optionally, non-amine polyurethane catalysts are used. Typical of such catalysts are organometallic compounds of bismuth, lead, tin, titanium, antimony, uranium, cadmium, cobalt, thorium, aluminum, mercury, zinc, nickel, cerium, molybdenum, vanadium, copper, manganese, zirconium, and the like. Included as illustrative are bismuth nitrate, lead 2-ethylhexoate, lead benzoate, ferric chloride, antimony trichloride and antimony glycolate. A preferred organo-tin class includes the stannous salts of carboxylic acids such as stannous octoate, stannous 2-ethylhexoate, stannous laurate, and the like, as well as dialkyl tin salts of carboxylic acids such as dibutyl tin diacetate, dibutyl tin dilaurate, dioctyl tin diacetate, and the like.

In the preparation of polyisocyanurate foams, trimerization catalysts are used for the purpose of converting the blends in conjunction with excess A component to polyisocyanurate-polyurethane foams. The trimerization catalysts employed can be any catalyst known to one skilled in the art, including, but not limited to, glycine salts and tertiary amine trimerization catalysts and alkali metal carboxylic acid salts and mixtures of the various types of catalysts. Preferred species within the classes are potassium acetate, potassium octoate, and N-(2-hydroxy-5-nonylphenol)methyl-N-methylglycinate.

Dispersing agents, cell stabilizers, and surfactants can be incorporated into the present blends. Surfactants, which are, generally, polysiloxane polyoxyalkylene block co-polymers, such as those disclosed in U.S. Pat. Nos. 2,834,748, 2,917,480, and 2,846,458, which are incorporated herein by reference.

Other optional additives for the blends can include flame retardants such as tris(2-chloroethyl)phosphate, tris(2-chloropropyl)phosphate, tris(2,3-dibromopropyl)phosphate, tris(1,3-dichloropropyl)phosphate, diammonium phosphate, various halogenated aromatic compounds, antimony oxide, aluminum trihydrate, polyvinyl chloride, and the like. Other optional ingredients can include from 0 to about 3 percent water, which chemically reacts with the isocyanate to produce carbon dioxide. This carbon dioxide acts as an auxiliary blowing agent.

Also included in the mixture are blowing agents or blowing agent blends as disclosed in this invention. Generally speaking, the amount present in the blended mixture are dictated by the desired foam densities of the final polyurethane or polyisocyanurate foams produces. The proportions in parts by weight of the total blowing agent blend can fall within the range of from 1 to about 45 parts of blowing agent per 100 parts of polyol, preferably from about 4 to about 30 parts.

The polyurethane foams produced can vary in density from about 0.5 pound per cubic foot to about 40 pounds per cubic foot, preferably from about 1.0 to 20.0 pounds per cubic foot, and most preferably from about 1.5 to 6.0 pounds per cubic foot. The density obtained is a function of how much of the blowing agent or blowing agent mixture disclosed in this invention is present in the A and/or B components, or alternatively added at the time the foam is prepared.

### Foams and Foamable Compositions:

Certain embodiments of the present invention involve a foam comprising a polyurethane-, polyisocyanurate-, or phenolic-based cell wall and a cell gas disposed within at least a portion of the cells, wherein the cell gas comprises the azeotrope-like mixture described herein. In certain embodiments, the foams are extruded thermoplastic foams. Preferred foams have a density ranging from about 0.5 pounds per cubic foot to about 60 pounds per cubic foot, preferably from about 1.0 to 20.0 pounds per cubic foot, and most preferably from about 1.5 to 6.0 pounds per cubic foot. The foam density is a function of how much of the blowing agent or blowing agent mixture (i.e., the azeotrope-like mixture and any auxiliary blowing agent, such as carbon dioxide, chemical blowing agent or other co-blowing agent is present in the molten polymer). These foams are generally rigid but can be made in various grades of softness to suit the end use requirements. The foams can have a closed cell structure, an open cell structure or a mixture of open and closed cells, with closed cell structures being preferred. These foams are used in a variety of well known applications, including but not limited to thermal insulation, flotation, packaging, void filling, crafts and decorative, and shock absorption.

In other embodiments, the invention provides foamable compositions. The foamable compositions of the present invention generally include one or more components capable of forming foam, such as polyurethane, polyisocyanurate, and phenolic-based compositions, and a blowing agent comprising at least one azeotrope-like mixture described herein. In certain embodiments, the foamable composition comprises thermoplastic materials, particularly thermoplastic polymers and/or resins. Examples of thermoplastic foam components include polyolefins, such as polystyrene (PS), polyethylene (PE), polypropylene (PP) and polyethyleneterepthalate (PET), and foams formed there from, preferably low-density foams. In certain embodiments, the thermoplastic foamable composition is an extrudable composition.

In certain embodiments, provided is a method for producing such foams. It will be appreciated by those skilled in the art, especially in view of the disclosure contained herein, that the order and manner in which the blowing agent is formed and/or added to the foamable composition does not generally affect the operability of the present invention. For example, in the case of extrudable foams, it is possible to mix in advance the various components of the blowing agent. In certain embodiments, the components of the foamable composition are not mixed in advance of introduction to the extrusion equipment or are not added to the same location in the extrusion equipment. Thus, in certain embodiments it may be desired to introduce one or more components of the blowing agent at first location in the extruder, which is upstream of the place of addition of one or more other components of the blowing agent, with the expectation that the components will come together in the extruder and/or operate more effectively in this manner. In certain other embodiments, two or more components of the blowing agent are combined in advance and introduced together into the foamable composition, either directly or as part of premix which is then further added to other parts of the foamable composition.

### Refrigerants and Heat Transfer Systems:

Another embodiment of the present invention relates to refrigerant compositions comprising the azeotrope-like compositions described herein. The refrigerant compositions of the present invention may be used in any of a wide variety of refrigeration systems including air-conditioning, refrigeration, heat-pump systems, and the like. In certain preferred embodiments, the compositions of the present invention are used in refrigeration systems originally designed for use with a CFC, HCFC, or HFC refrigerant, such as, for example, HFC-134a and the like. The preferred compositions of the present invention tend to exhibit many of the desirable characteristics of HFC-134a and other HFC refrigerants, including non-flammability, and a GWP that is as low as, or lower than, that of conventional HFC refrigerants. In addition, the relatively constant boiling nature of the compositions of the present invention makes them more desirable than certain conventional HFCs for use as refrigerants in many applications.

In certain embodiments, the compositions of the present invention may be used to retrofit refrigeration systems containing HFC, HCFC, and/or CFC refrigerants and lubricants used conventionally therewith, such as mineral oils, silicone oils, and the like. Preferably, the present methods involve recharging a refrigerant system that contains a refrigerant to be replaced and a lubricant, the method comprising the steps of (a) removing at least a substantial portion of the refrigerant to be replaced from the refrigeration system while retaining a substantial portion of the lubricant in said system; and (b) introducing to the system a refrigerant comprising an azeotrope-like mixture as described herein. As used herein, the term “substantial portion” refers generally to a quantity of lubricant or refrigerant which is at least about 50% (by weight) of the quantity of lubricant or refrigerant, respectively, contained in the refrigeration system prior to removal of the previous, less environmentally friendly refrigerant. Preferably, the substantial portion of lubricant or refrigerant in the system according to the present invention is a quantity of at least about 60% of the lubricant or refrigerant, respectively, contained originally in the refrigeration system, and more preferably a quantity of at least about 70%. As used herein the term “refrigeration system” refers generally to any system or apparatus, or any part or portion of such a system or apparatus, which employs a refrigerant to provide cooling. Such refrigeration systems include, for example, air conditioners, electric refrigerators, chillers, transport refrigeration systems, commercial refrigeration systems and the like.

Any of a wide range of known methods can be used to remove refrigerants to be replaced from a refrigeration system while removing less than a major portion of the lubricant contained in the system. For example, because refrigerants are quite volatile relative to traditional hydrocarbon-based lubricants (the boiling points of refrigerants are generally less than 10° C. whereas the boiling points of mineral oils are generally more than 200° C.), in embodiments wherein the lubricant is a hydrocarbon-based lubricant, the removal step may readily be performed by pumping refrigerants in the gaseous state out of a refrigeration system containing liquid state lubricants. Such removal can be achieved in any of a number of ways known in the art, including, the use of a refrigerant recovery system, such as the recovery system manufactured by Robinair of Ohio. Alternatively, a cooled, evacuated refrigerant container can be attached to the low pressure side of a refrigeration system such that the gaseous refrigerant is drawn into the evacuated container and removed. Moreover, a compressor may be attached to a refrigeration system to pump the refrigerant from the system to an evacuated container. Those of ordinary skill in the art also will be readily able to remove the lubricants from refrigeration systems and to provide a refrigeration system having therein a lubricant and refrigerant according to the present invention.

Any of a wide range of methods for introducing the present refrigerant compositions to a refrigeration system can be used in the present invention. For example, one method comprises attaching a refrigerant container to the low-pressure side of a refrigeration system and turning on the refrigeration system compressor to pull the refrigerant into the system. In such embodiments, the refrigerant container may be placed on a scale such that the amount of refrigerant composition entering the system can be monitored. When a desired amount of refrigerant composition has been introduced into the system, charging is stopped. Alternatively, a wide range of charging tools, known to those of skill in the art, are commercially available. Accordingly, in light of the above disclosure, those of skill in the art will be readily able to introduce the refrigerant compositions of the present invention into refrigeration systems according to the present invention without undue experimentation.

According to certain other embodiments, the present invention provides refrigeration systems comprising a refrigerant of the present invention and methods of producing heating or cooling by condensing and/or evaporating a composition of the present invention. In certain preferred embodiments, the methods for cooling an article according to the present invention comprise condensing a refrigerant composition comprising an azeotrope-like composition of the present invention and thereafter evaporating said refrigerant composition in the vicinity of the article to be cooled. Certain preferred methods for heating an article comprise condensing a refrigerant composition comprising an azeotrope-like composition of the present invention in the vicinity of the article to be heated and thereafter evaporating said refrigerant composition. In light of the disclosure herein, those of skill in the art will be readily able to heat and cool articles according to the present inventions without undue experimentation.

### Sprayable Compositions:

In a preferred embodiment, the azeotrope-like compositions of this invention may be used as solvents in sprayable compositions, either alone or in combination with other known propellants. The solvent composition comprises, more preferably consists essentially of, and, even more preferably, consists of the azeotrope-like compositions of the invention. In certain embodiments, the sprayable composition is an aerosol.

In certain preferred embodiments, provided is a sprayable composition comprising a solvent as described above, an active ingredient, and optionally, other components such as inert ingredients, solvents, and the like. Suitable active materials to be sprayed include, without limitation, cosmetic materials such as deodorants, perfumes, hair sprays, cleansers, defluxing agents, and polishing agents as well as medicinal materials such as anti-asthma and anti-halitosis medications. The term medicinal materials is used herein in its broadest sense to include any and all materials which are, or at least are believe to be, effective in connection with therapeutic, diagnostic, pain relief, and similar treatments, and as such would include for example drugs and biologically active substances.

### Solvents and Cleaning Compositions:

In another embodiment of the invention, the azeotrope-like compositions described herein can be used as a solvent in cleaning various soils such as mineral oil, rosin based fluxes, lubricants, etc., from various substrates by wiping, vapor degreasing, or other means. In certain preferred embodiments, the cleaning composition is an aerosol.

### EXAMPLES

The invention is further illustrated in the following example which is intended to be illustrative, but not limiting in any manner.

### Example 1

An ebulliometer consisting of vacuum jacketed tube with a condenser on top which is further equipped with a Quartz Thermometer is used. About 19.79 g of HFO-1234yf is charged to the ebulliometer and then 1243zf is added in small, measured increments. Temperature depression is observed at 14.3 psia when 1243zf is added to 1234yf, indicating a binary minimum boiling azeotrope is formed. From greater than about 0 to about 5 weight percent 1234yf, the boiling point of the composition stays below or around the boiling point of 1234yf. The normal boiling temperature of 1243zf is about -26° C. The binary mixtures shown in Table 1 were studied and the boiling point of the compositions did not go above the boiling point of HFO-1234yf. The compositions exhibit azeotrope and/or azeotrope-like properties over this range.

TABLE 1

| T (C.) | Wt. % 1234yf | Wt. % 1243zf |
|---|---|---|
| −29.85 | 100.0 | 0.00 |
| −29.96 | 99.25 | 0.75 |
| −29.94 | 98.75 | 1.25 |
| −29.85 | 95.79 | 4.21 |
| −29.72 | 92.09 | 7.91 |
| −29.51 | 85.82 | 14.18 |
| −29.39 | 82.36 | 17.64 |
| −29.24 | 78.04 | 21.96 |
| −29.11 | 74.82 | 25.18 |
| −28.84 | 67.94 | 32.06 |
| −28.67 | 63.86 | 36.14 |

### Example 2

Approximately 2 g of 3,3,3-trifluoropropene (1243zf) were dissolved in 98 g of 2,2,2,3-tetrafluoropropene (1234yf) to form a homogeneous azeotrope mixture. This experiment was done at 25° C., and at 14.6 psia.

### Example 3

Binary compositions containing solely 3,3,3-trifluoropropene (1243zf) and 2,2,2,3-tetrafluoropropene (1234yf) are blended to form homogeneous azeotrope mixtures at different compositions. The vapor pressures of the mixtures are measured at about 45 and 60° C. and the following results are noticed. Table 2 shows the vapor pressure measurement of 1234yf and 1243zf as a function of composition of weight percent 1243zf at constant temperatures of about 45 and 60° C.

TABLE 2
P-T-X of 1234yf/1243zf System

Pressure (Psia)

| Wt. % 1243zf | T = 44.5° C. | T = 59.6° C. |
|---|---|---|
| 0.0 | 162.49 | 231.12 |
| 1.65 | 165.43 | 231.63 |
| 2.85 | 165.58 | 231.92 |
| 6.78 | 164.07 | 230.80 |
| 14.42 | 162.62 | 229.49 |

The data also show that the mixture is an azeotrope since the vapor pressures of mixtures of 1234yf and 1243zf are higher, at all indicated blend proportions, than 1234yf and 1243zf alone.

### Example 4

The azeotropic composition of the 1234yf/1243zf mixture is also verified by Vapor-Liquid-Equilibrium (VLE) experiment. Approximately 6.6 g of 1243zf are dissolved in 133.9 g of 1234yf to form a homogeneous mixture (4.67 wt. % 1243zf) at 23° C. The second mixture was prepared with the composition of 97.8 wt. % 1234yf and 2.2 wt. % 1243zf. The liquid and vapor compositions of the two mixtures were sampled at temperatures of about 45 and 55° C. The results are shown in Table 3 and indicate that the mixture of 1234yf/1243zf is azeotropic like at the experimental conditions.

TABLE 3
VLE of 1234yf/1243zf at 45 and 55° C.

| | | Compositions (wt. %) | | Liquid (L) or |
|---|---|---|---|---|
| T (° C.) | P (psia) | 1234yf | 1243zf | Vapor (V) |
| 44.6 | 165.5 | 94.90 | 5.10 | L |
| 44.6 | 165.5 | 94.90 | 5.10 | V |
| 44.6 | 165.5 | 94.90 | 5.10 | L |
| 44.6 | 165.5 | 94.88 | 5.12 | V |
| 54.9 | 209.6 | 94.85 | 5.15 | L |
| 54.9 | 209.6 | 94.86 | 5.14 | V |
| 54.9 | 209.7 | 94.86 | 5.14 | L |
| 54.9 | 209.7 | 94.85 | 5.15 | V |
| 44.6 | 165.8 | 97.76 | 2.24 | L |
| 44.6 | 165.8 | 97.97 | 2.03 | V |
| 44.6 | 165.8 | 97.76 | 2.24 | L |
| 44.6 | 165.8 | 97.95 | 2.05 | V |
| 54.9 | 209.9 | 97.79 | 2.21 | L |
| 54.9 | 209.9 | 97.97 | 2.03 | V |
| 54.9 | 209.9 | 97.81 | 2.19 | L |
| 54.9 | 209.9 | 97.94 | 2.06 | V |

### Example 5

A distillation column was used to determine the azeotropic composition of a mixture of 1234yf and 1243zf at various temperatures. The distillation column consists of a 1 L reboiler connected to a monel column which is 4 ft. long and has a 1 in. internal diameter. The column is packed with monel Heli-Pak high efficiency packing. The condenser is maintained at the desired temperature by circulating a propylene glycol water mix which is thermostated by means of a constant temperature bath. Vapor samples are taken from the overhead of the condenser to determine the azeotropic composition.

Initially a mixture of 93 wt % 1234yf and 7 wt % 1243zf were charged to the reboiler. The condenser temperature was controlled at temperatures between 14 and 66° C. At each condition the column was operated a full reflux until thermal equilibrium had been achieved. Once equilibrium had been established a vapor overhead sample was taken and analyzed by GC. The following Table 4 shows the results of the analysis. This indicates that an azeotrope of 1234yf/1243zf is formed over all of the temperatures tested. The azeotrope shifts from about 2 wt % 1243zf at 14° C. to about 5 wt % 1243zf at 66° C.

TABLE 4
Distillation of 1234yf/1243zf at temperature between 14 and 66° C.

| Temp, ° C. | 1243zf, wt % | 1234yf, wt % |
|---|---|---|
| 31 | 3.1 | 96.9 |
| 32 | 3.1 | 96.9 |
| 14 | 2.3 | 97.7 |
| 14 | 2.5 | 97.5 |
| 50 | 3.2 | 96.8 |
| 51 | 3.6 | 96.4 |
| 65 | 4.0 | 96.0 |
| 66 | 4.7 | 95.3 |

1.-45. (canceled)

46. A composition comprising an azeotrope-like mixture consisting essentially of 2,3,3,3-tetrafluoropropene and 3,3,3-trifluoropropene.

47. The composition of claim 46 consisting essentially of from about 60 to less than 100 weight percent 2,3,3,3-tetrafluoropropene and from greater than 0 to about 40 weight percent 3,3,3-trifluoropropene.

48. The composition of claim 47 consisting essentially of from about 85 to less than 100 weight percent 2,3,3,3-tetrafluoropropene and from greater than 0 to about 15 weight percent 3,3,3-trifluoropropene.

49. The composition of claim 48 consisting essentially of from about 95 to less than 100 weight percent 2,3,3,3-tetrafluoropropene and from greater than 0 to about 5 weight percent 3,3,3-trifluoropropene.

50. The composition of claim 49 consisting essentially of from about 95.8 to about 99.9 weight percent 2,3,3,3-tetrafluoropropene and from greater than 0.1 to about 4.2 weight percent 3,3,3-trifluoropropene.

51. The composition of claim 49 wherein said composition has a boiling point of from about −30° C. to about 66° C. at a pressure of from about 14 psia to about 230 psia.

52. The composition of claim 50 wherein said composition is maintained at a pressure of from about 162.5±0.5 psia to about 166.6±0.5 psia.

53. The composition of claim 51 wherein said composition is maintained at a pressure of about 14.3±0.5 psia.

54. The composition of claim 51 wherein said composition is maintained at a pressure of from about 231.1±0.5 psia to about 231.9±0.5 psia.

55. The composition of claim 46 further comprising one or more refrigerant additives selected from the group consisting of lubricants, stabilizers, metal passivators, corrosion inhibitors, flammability suppressants, compatibilizers, surfactants, and solubilizing agents.

56. The composition of claim 46 further comprising one or more refrigerant lubricants selected from the group consisting of mineral oil, polyalkylene glycol, polyalkylene glycol ester, polyvinyl ethers, and polyol esters.

57. The composition of claim 46 comprising further comprising an active ingredient selected from the group consisting of cosmetics, polishing agents, medicaments, cleaners, fire retarding agents, colorants, and chemical sterilants, wherein said composition is sprayable and said azeotropic mixture is a carrier for said active ingredient.

58. The composition of claim 57 wherein said composition is an aerosol.

59. The composition of claim 46 further comprising at least one blowing agent additive selected from the group consisting of co-blowing agents, fillers, vapor pressure modifiers, flame suppressants, and stabilizers.

60. The composition of claim 46 further comprising at least one blowing agent additive selected from the group consisting of co-blowing agents, fillers, vapor pressure modifiers, flame suppressants, and stabilizers.

61. The composition of claim 46 further comprising a thermoplastic or thermosetting resin selected from the group consisting of polystyrene, polyethylene, polypropylene, polyvinyl chloride, polytheyeneterephthalate, polyurethane, polyisocyanurate, phenolic, epoxy, or combinations thereof, wherein said composition is capable of producing a foam.

62. A method for purifying a hydrofluoroolefin comprising:
a. providing a composition comprising 2,3,3,3-tetrafluoropropene and 3,3,3-trifluoropropene;
b. distilling said composition to produce a first stream enriched in either said 2,3,3,3-tetrafluoropropene or said 3,3,3-trifluoropropene and a second stream comprising an azeotrope-like mixture according to claim 46.

63. The method of claim 62 further comprising the steps of:
c. breaking said azeotrope-like composition by subjecting said azeotrope-like composition to at least one separation method selected from the group consisting of swing distillation, extractive distillation, azeotrope distillation, evaporation, and phase separation; and
d. separating said broken azeotrope-like composition into a first component rich in 2,3,3,3-tetrafluoropropene and a second component rich in 3,3,3-trifluoropropene.
